SEQUENCE LISTING (1 sequence)

SEQ ID NO: 1 - protein, 282 aa, human [sequence omitted]

(19) United States
(12) Patent Application Publication
(10) Pub. No.: US 2024/0216483 A1
(43) Pub. Date: Jul. 4, 2024

(54) THERAPEUTIC REGIMEN WITH DNASE

(71) Applicant: PERI-NESS TECHNOLOGIES LTD, Giv'at Shmuel (IL)

(72) Inventors: Moshe MIZRAHY, Tel Aviv (IL); Dmitry GENKIN, Schulstrasse (DE)

(73) Assignee: PERI-NESS TECHNOLOGIES LTD, Giv'at Shmuel (IL)

(21) Appl. No.: 18/562,803

(22) PCT Filed: May 24, 2022
(86) PCT No.: PCT/IL2022/050541

§ 371 Date: Nov. 20, 2023

Related U.S. Application Data
(60) Provisional application No. 63/192,204, filed on May 24, 2021.

(51) Int. Cl. A61K 38/46; A61P 15/08

(52) U.S. Cl. CPC A61K 38/465; A61P 15/08; C12Y 301/21001

(57) ABSTRACT

The invention provides methods for induction of pregnancy in a couple in need thereof wherein the male subject in said couple has been diagnosed as sub-fertile, said method comprising administering systemically to said male subject a pharmaceutical composition comprising a human DNase.

### TECHNOLOGICAL FIELD

The present invention generally relates to methods for induction of pregnancy in couples wherein the male subject has been diagnosed as sub-fertile.

### BACKGROUND

Most couples are able to conceive spontaneously within 12 months of having regular unprotected intercourse. Sub-fertility is defined as a delay in conceiving and may result from pathologies in either the female or the male partner.

Male fertility status is evaluated by a semen analysis providing information on the number of spermatozoa, their motility and morphology.

Idiopathic oligoasthenoteratozoospermia is the commonest cause of male subfertility. Although sexual function is normal, there is a reduced count of mainly dysfunctional spermatozoa. Reduced fertilizing capacity is related to raised concentrations of reactive oxygen species in semen, which may damage the cell membrane. Abnormal sperm morphology—an indicator of deranged sperm production or maturation—is also associated with reduced fertilizing capacity. Less common types of male subfertility are caused by testicular or genital tract infection, disease, or abnormalities. Systemic disease, external factors (such as drugs, lifestyle, etc.), or combinations of these also result in male subfertility. Male subfertility is rarely caused by endocrine deficiency (Hirsh A. BUM 2003; 327 (7424): 1165).

U.S. Pat. No. 9,149,513 provides methods for treating male sub-fertility by administering a pharmaceutical composition comprising DNase. The male subjects received four times a day 25 mg of bovine DNase I by intramuscular (IM) injection for a period of 7-10 days.

### General Description

In a first of its aspects, the present invention provides a method for induction of pregnancy in a couple in need thereof wherein the male subject in said couple has been diagnosed as sub-fertile, said method comprising administering systemically to said male subject a pharmaceutical composition comprising between about 50 and about 1000 μg/kg human DNase.

In one embodiment, said pharmaceutical composition is administered once a week, once every three days, once every two days, once a day, twice a day, or three times a day.

In one embodiment, said pharmaceutical composition is administered for a treatment course period sufficient to achieve fresh spermatid maturation in said male subject.

In one embodiment, said administration is for a treatment course period of between about 10 days and about 3 months, or between about 10 days and 16 days.

In one embodiment, the pharmaceutical composition is administered intravenously (IV), subcutaneously (SC), intramuscularly (IM), by inhalation or orally.

In one embodiment, the human male subject has an idiopathic reduction in semen quality.

In one embodiment, the human male subject has azoospermia, oligozoospermia, mild or severe isolated teratozoospermia, asthenozoospermia, or oligoteratoasthenospermia (OTA).

In one embodiment, the human male subject is a male partner of a couple that had been failing to conceive or had been miscarrying for two years or more.

In one embodiment, the human male subject is a male partner of a couple that had been failing to conceive in at least one assisted reproductive technology (ART) procedure.

In one embodiment, said couple had been failing to conceive in at least two assisted reproductive technology (ART) procedures.

In one embodiment, the human female subject of said couple was not diagnosed with infertility.

In one embodiment, said ART is intracytoplasmic sperm injection (ICSI), intracytoplasmic morphologically selected sperm injection (IMSI), or intrauterine insemination (IUI).

In one embodiment, the human male subject has a low sperm viability and/or motility and/or concentration.

In one embodiment, the human male subject has high blood cell-free DNA levels.

In one embodiment, the sperm of said human male subject is used in ART.

In one embodiment, said ART is intracytoplasmic sperm injection (ICSI), intracytoplasmic morphologically selected sperm injection (IMSI), or intrauterine insemination (IUI).

In one embodiment, administration of said pharmaceutical composition results in improvement of at least 35% in one or more sperm quality parameters selected from the group consisting of total sperm count, sperm concentration, sperm motility, sperm motility quality or average speed, sperm morphology, sperm viability and sperm chromatin stability.

In one embodiment, sperm of said human male subject is sampled within 1 to 14 days following completion of said treatment course period.

In one embodiment, the sperm of said human male subject is sampled within 1 to 5 days following completion of said treatment course period.

In one embodiment, completion of the treatment course period with said pharmaceutical composition is synchronized with ovulation in the female subject in said couple.

In one embodiment, ovulation subsides 1 to 14 days following completion of the treatment course period.

In one embodiment, ovulation subsides 1 to 5 days following completion of the treatment course period.

In one embodiment, the method of the invention further comprises administration of an additional therapeutic agent.

In another aspect, the present invention provides a pharmaceutical composition comprising DNase and a pharmaceutically acceptable carrier for use in a method for induction of pregnancy in a couple in need thereof wherein the human male subject in said couple has been diagnosed as sub-fertile, wherein said method comprising administering said pharmaceutical composition systemically to a human subject in need thereof and wherein said pharmaceutical composition comprises between about 50 and about 1000 μg/kg DNase.

In one embodiment, said pharmaceutical composition comprises an additional therapeutic agent.

In some embodiments, said DNase is pegylated DNAse or PASylated DNAse

In some embodiments, said DNase is selected from a group consisting of DNase I, DNase I L1, DNase I L2, DNase I L3, DNase II, DNase IIα, DNase II β, DNase X, DNase γ, Caspase-activated DNase (CAD), Endonuclease G (ENDOG), Granzyme B (GZMB), phosphodiesterase I, lactoferrin, acetylcholinesterase, and variants thereof that maintain the DNAse activity.

In one embodiment, said DNase is human DNase I.

In one embodiment, said human DNAse I comprises an amino acid sequence having at least 90% sequence identity to SEQ ID NO: 1, or to the mature DNAse I enzyme without the signal peptide.

In one embodiment, said DNase I is human recombinant DNase I, or isolated natural human DNase I.

In one embodiment, said DNase I is human DNase I, comprising an amino acid sequence having at least 90% sequence identity to SEQ ID NO: 1, and said human DNase I is injected intravenously for 14-16 days at a dose of 250 μg/kg/day and the sperm of said human male subject is sampled within 1 to 5 days following completion of said treatment course period.

In one embodiment, said DNase I is human DNase I, comprising an amino acid sequence having at least 90% sequence identity to SEQ ID NO: 1, and said human DNase I is injected intravenously for 14-16 days at the dose of 250 μg/kg/day and ovulation subsides 1 to 5 days following completion of the treatment course period.

### BRIEF DESCRIPTION OF THE DRAWINGS

For better understanding the subject matter that is disclosed herein and to exemplify how it may be carried out in practice, embodiments will now be described, by way of non-limiting example only, with reference to the accompanying drawings, in which:

FIG. 1 is an outline of the amino acid sequence of human DNase I (denoted herein as SEQ ID NO. 1).

FIG. 2 is a graph showing the percent of oval-shape sperm cells out of the whole population of cells considered to be sperm cells, over time (days), observed using high power morphological examination (MSOME). The rectangle represents the 16-day period of DNase I treatment.

FIG. 3 is a graph showing the percent of amorphic sperm cells out of the whole population of cells considered to be sperm cells, over time (days), observed using high power morphological examination (MSOME). The rectangle represents the 16-day period of DNase I treatment.

FIG. 4 is a graph showing the percent of oval-shape sperm cells out of the whole population of cells considered to be sperm cells, over time (days), observed using high power morphological examination (MSOME). The rectangle represents the 16-day period of DNase I treatment.

FIG. 5 is a graph showing the number of round and amorphic sperm cells, over time (days), observed using high power morphological examination (MSOME). The rectangle represents the 16-day period of DNase I treatment.

FIG. 6 is a graph showing the number of normal sperm cells, over time (days), as determined by Halosperm analysis.

FIG. 7 is a graph showing the percent of normal sperm cells forms according to WHO routine morphology criteria out of the whole population of cells considered to be sperm cells, over time (days). The rectangle represents the 16-day period of DNase I treatment. The line represents the normal level.

FIG. 8 is a graph showing the percent of sperm cells with head defects according to WHO routine morphology criteria out of the whole population of cells considered to be sperm cells, over time (days). The rectangle represents the 16-day period of DNase I treatment. The line represents the normal level.

FIG. 9 is a graph showing the number of percent of normal sperm cells, over time (days), as determined by Halosperm analysis. The rectangle represents the 16-day period of DNase I treatment. The line represents the normal level.

FIG. 10 is a graph showing the kinetics of DNase I activity mKU (Kunitz)/μl following injection of 125 μg/kg recombinant human DNAse I (rhDNase I) over time (hours), as determined by SRED test. A Kunitz unit is defined as the amount of enzyme added to 1 mg/ml salmon sperm DNA that causes an increase in absorbance of 0.001 per minute at the wavelength of 260 nm when acting upon highly polymerized DNA at 25° C. in a 0.1 M NaOAc (pH 5.0) buffer.

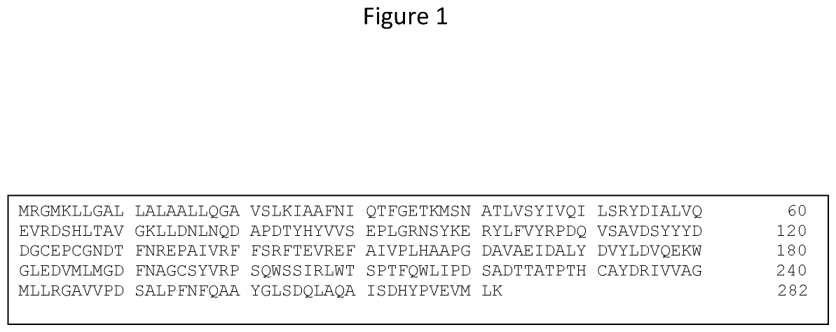

### DETAILED DESCRIPTION OF EMBODIMENTS

The present invention provides a treatment regimen for induction of pregnancy in couples wherein the male subject has been diagnosed as sub-fertile. The treatment regimen comprises administering a clinical grade pharmaceutical composition comprising human deoxyribonuclease (DNase), e.g., DNase I.

As shown in the Example below, administration of DNase I resulted in successful pregnancies in previously non-conceiving couples. The treatment caused an improvement in sperm cell DNA stability as observed in the Halosperm test and improvement in sperm nuclear morphology as observed by MSOME analysis.

Without wishing to be bound by theory, these analyses represent two processes mediated by the recombinant human DNAse I (rhDNase) treatment.

The improvement in sperm cell DNA stability, as measured by % DFI in the Halosperm test, was rapid and transient, and was evident mainly within the 16-day treatment time frame (a short-term effect).

The improvement in sperm morphology was observed later, reaching maximum values on day 24, 8 days after treatment has been completed (long term effect).

It is suggested that sperm cells passing through the epididymis (process duration: 8 days) may be prone to apoptosis induced by cell free DNA, and that DNase treatment may prevent or limit this damage, thus improving % DFI in the Halosperm test.

It is further suggested that DNase also targets sperm cells during spermatid maturation in spermiogenesis which lasts for 16 days and occurs before epididymal transport.

In this process sperm cells gain their required morphology, and it is postulated that the presence of cell free DNA may impair their morphology. DNase treatment may prevent or limit these effects.

The results from the clinical studies in sub-fertile males suggest that cell-free DNA is associated with sperm cell damage, and that DNase treatment has the potential to significantly improve semen quality and lead to successful pregnancies.

Therefore, in a first of its aspects the present invention provides a method for induction of pregnancy in a couple in need thereof wherein the male subject in said couple has been diagnosed as sub-fertile, said method comprising administering systemically to said male subject a pharmaceutical composition comprising between about 50 μg/kg and about 1000 μg/kg, or between about 50 μg/kg and about 350 μg/kg human DNase, e.g., 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, or 350 μg/kg. In one embodiment, the pharmaceutical composition comprises 125 μg/kg.

Induction of pregnancy as used herein refers to achieving a successful pregnancy in previously non-conceiving couples.

The terms “subject” or “patient” are used interchangeably herein and refer to a male subject that may benefit from the present invention. At times it is also referred to as “a male partner” in a couple. In one specific embodiment the subject is human.

The term “subject in need thereof” in the context of the present invention inter alia refers to human male subjects suffering from a disease or disorder that results in sub-fertility, as defined herein. The term “couple in need thereof” in the context of the present invention inter alia refers to a human couple (male and female) which show a reduced ability to conceive, as defined herein.

As used herein, the term “male sub-fertility” or “sub-fertile male” is used in its broadest sense and refers to any condition which is characterized by documented idiopathic reduction in semen quality.

Examples include, but are not limited to:
Oligozoospermia—reduced sperm numbers. Mild to moderate: 5-20 million/ml of semen. Severe: <5 million/ml of semen.
Asthenozoospermia—reduced sperm motility.
Teratozoospermia (mild or severe)—increased abnormal forms of sperm.
Azoospermia and oligoteratoasthenospermia (OTA).
Semen with high DFI (DNA Fragmentation Index) values

DNA fragmentation can be measured using methods known to those skilled in the art such as gel electrophoresis or the Halosperm test. Halosperm is based on the Sperm Chromatin Dispersion (SCD) technique and may be performed using in vitro diagnostic kit [Halotech DNA, Spain] that controls DNA denaturation process to facilitate the subsequent removal of the proteins contained in each spermatozoon. In this way, normal spermatozoa create halos formed by loops of DNA at the head of the sperm, which are not present in those with damaged DNA.

In certain embodiments the term “reduced” is meant to refer to a reduction of at least about 50%, 60%, 70%, 80%, or 90% in sperm numbers or motility as compared to fertile males.

The term also encompasses male partners of couples that had been attempting to conceive for more than two years and had previous failures in assisted reproductive technology (ART) with no diagnosed female factor of infertility.

In certain embodiments in accordance with the invention, administration of human DNAse I results in improvement of at least 35% in one or more semen quality parameters selected from: total count, concentration, motility, motility quality or average speed, morphology (Kruger or WHO), viability (eosin staining) and sperm chromatin stability (Halosperm assay).

By the term “improvement” of semen quality parameters in the context of the present invention it is meant that the pharmaceutical composition of the invention increases the semen quality parameters as measured by any means known in the art, by at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100% as compared to these quality parameters in the absence of the pharmaceutical composition of the invention.

Systemic administration according to the present invention may be performed by any of the following routes: oral administration, intravenous, intramuscular, intraperitoneal, intrathecal, or subcutaneous injection.

In specific embodiments administration according to the present invention is performed intravenously.

The pharmaceutical compositions comprising DNAse may be administered to a subject in a single dose or in multiple doses.

In some embodiments the pharmaceutical composition in accordance with the invention comprises 50 μg/kg to about 1000 μg/kg DNAse.

The pharmaceutical composition may be administered multiple times, e.g., from a frequency of 5 administrations per day to a frequency of once every three days, or from a frequency of 3 administrations per day to a frequency of once every three days, namely 3 times a day, or twice a day, or once a day, or once every two days, or once every three days, or once a week.

The pharmaceutical composition may be administered for a length of time of at least 10 days, or for a length of time of between about 10 days and about 3 months, e.g., for between about 10 days and 16 days, or for 16 days. The time-period in which treatment is administered is also defined herein as the “treatment course period”.

In an embodiment, the pharmaceutical composition is administered for a treatment course period sufficient to achieve fresh spermatid maturation in said male subject. As used herein the term “fresh spermatid maturation” refers to the appearance of newly formed mature spermatozoa. Spermatids mature into spermatozoa during spermiogenesis which is the final stage of spermatogenesis. Therefore, in an embodiment, the pharmaceutical composition is administered for a treatment course period sufficient to achieve effective spermatozoa levels in said male subject. Effective refers herein to levels of spermatozoa sufficient to achieve pregnancy induction in the treated couple.

In one embodiment the method comprises administration of 250 μg DNAse I of the invention/kg body weight per day (i.e., 250 μg/kg/day).

In one embodiment the pharmaceutical composition is administered at a dose of 125 μg/kg body weight twice daily (12 hours apart, for example once in the morning and once in the evening) for 16 days.

As used herein the term “DNase” refers to any enzyme capable of cleaving DNA and represents a protein family. Accordingly, the term includes but is not limited to, any enzyme selected from the group consisting of DNase I, DNase I like 1 (DNaseIL1), DNase I like 2 (DNase IL2), DNase I like 3 (DNase IL3), DNase II, DNase IIα, DNase II β, DNase X, DNase γ, Caspase-activated DNase (“CAD”), Endonuclease G (“ENDOG”), Granzyme B (“GZMB”), phosphodiesterase I, lactoferrin, acetyl-cholinesterase, and variants thereof that maintain the DNAse activity, including, but not limited to one or more mutations in the actin binding site thereof.

In one embodiment, the DNAse is DNAse I. DNAseI preferentially cleaves protein-free DNA, and its activity is inhibited upon binding to monomeric actin. DNAse I is sensitive to physiological salt concentrations. Furthermore, DNAse I is glycosylated at N40 (corresponds to N18 in the mature enzyme without signal peptide) and N128 (N106), which makes the enzyme resistant to inactivation by serum proteases.

DNase I may be recombinantly produced (e.g., Pulmozyme) or be a natural isolated DNAse I.

It is appreciated that the term “purified” or “isolated” refers to molecules, e.g., DNAse that are removed from their natural environment, isolated, or separated. An “isolated DNAse” is therefore a purified enzyme. As used herein, the term “purified” or “to purify” also refers to the removal of contaminants from a sample.

The DNAse I in accordance with the invention may be obtained from a variety of species including humans, primates, and rodents. In a preferred embodiment, the DNAse I of the invention is human DNAse I. Human DNase I may be obtained from various commercial sources.

The DNAse of the invention may also be a modified version of the enzyme in which physical, enzymatical, and/or pharmacodynamic properties were enhanced (see for example U.S. Pat. No. 11,046,943).

Therefore, the present invention also encompasses administration of variants of human DNAse. The variants may include mutations which do not alter the activity of the enzyme.

By the term “activity of the enzyme” it is meant the ability of the enzyme to cleave DNA (e.g., a DNA fragmentation activity), and preferably to affect semen quality parameters as defined herein. The activity of the enzyme can be measured in vivo or in vitro using methods well known in the art, e.g., as described in the Examples below.

By the term “variant” it is meant sequences of amino acids different from the sequence of DNAse I specifically identified herein, in which one or more amino acid residues are deleted, substituted, or added.

It should be appreciated that by the term “added”, as used herein it is meant any addition of amino acid residues to the sequence described herein.

Variants encompass various amino acid substitutions. An amino acid “substitution” is the result of replacing one amino acid with another amino acid which has similar or different structural and/or chemical properties. Amino acid substitutions may be made based on similarity in polarity, charge, solubility, hydrophobicity, hydrophilicity, and/or the amphipathic nature of the residues involved.

Typically, variants encompass conservative amino acid substitutions. Conservative substitution tables providing functionally similar amino acids are well known in the art. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

Each of the following eight groups contains other exemplary amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

Variants in accordance with the invention also encompass non-polar to polar amino acid substitutions and vice-versa.

As used herein, the term “amino acid” or “amino acid residue” refers to naturally occurring and synthetic amino acids, as well as amino acid analogs and amino acid mimetics that function similarly to the naturally occurring amino acids.

Variant sequences refer to amino acid sequences that may be characterized by the percentage of the identity of their amino acid sequences with the amino acid sequence of DNAse I described herein.

In some embodiments, variant sequences as herein defined refer to amino acid sequences of DNAse I, having a sequence of amino acids with at least 70% or 75% of sequence identity, around 80% or 85% of sequence identity, around 90%, 91%, 92%, 93%, 94%, 95%, 96%, 97%, 98% or 99% of sequence identity when compared to the sequence of DNAse I denoted herein in SEQ ID NO. 1 (also referred to herein as the wildtype sequence):

MRGMKLLGALLALAALLQGAVSLKIAAFNIQTFGETKMSNATLVSY
IVQILSRYDIALVQEVRDSHLTAVGKLLDNLNQDAPDTYHYVVSE
PLGRNSYKERYLFVYRPDQVSAVDSYYYDDGCEPCGNDTFNREPA
IVRFFSRFTEVREFAIVPLHAAPGDAVAEIDALYDVYLDVQEKWG
LEDVMLMGDFNAGCSYVRPSQWSSIRLWTSPTFQWLIPDSADTTA
TPTHCAYDRIVVAGMLLRGAVVPDSALPFNFQAAYGLSDQLAQAI
SDHYPVEVMLK

The similarity of amino acid sequences, i.e., the percentage of sequence identity can be determined via sequence alignments as known in the art. Such alignments can be carried out with several art-known algorithms, e.g., BLAST programs.

The first 22 amino acid residues marked in bold letters represent the signal peptide. The mature enzyme is defined as having the sequence denoted by SEQ ID NO. 1 without the signal peptide (i.e., without the first 22 amino acids as indicated above). As used herein, when referring to sequence identity with wild-type DNAse enzymes, sequences refer to mature enzymes lacking the signal peptide.

In some embodiments, the DNAse variant comprises an N-terminal or C-terminal fusion to a half-life extending moiety, such as albumin, transferrin, an Fc, or elastin-like protein.

In some embodiments, the DNAse variant comprises one or more polyethylene glycol (PEG) moieties, which may be conjugated at one or more of positions or at the C-terminus. The Dnase variant in such case is termed pegylated DNAse. Pegylation may be performed by any method known in the art. In other embodiments, the DNAse variant is PASylated for extending the plasma half-life of the enzyme. PASylation is performed by incorporating the small amino acid residues Pro, Ala and Ser (PAS) to the enzyme (see for example, Schlapschy et al protein Eng Des Sel 2013; 26(8):489-501). The Dnase variant in such case is termed PASylated DNAse.

In one aspect the invention provides pharmaceutical compositions.

The term “pharmaceutical composition” in accordance with the invention generally comprises DNAse as herein defined and a buffering agent, an agent which adjusts the osmolarity of the composition and optionally, one or more pharmaceutically acceptable carriers, excipients and/or diluents as known in the art.

As used herein the term “pharmaceutically acceptable carrier, excipient or diluent” refers to any one of inert, non-toxic, excipients, which essentially do not react with the DNAse I and is added thereto in order to give form or consistency to the pharmaceutical composition and to provide protection from degradation of the DNAse I and increase its survival outside and inside the body and to obtain penetration into the body and delivery into the body fluids and to facilitates distribution of the agent in the subject's body and delivery to the target site (e.g., to the cell free DNA).

The “pharmaceutically acceptable carrier, excipient or diluent” includes any solvent, dispersion medium, coating, antibacterial and antifungal agent, and the like, as known in the art. The carrier can be a solvent or dispersion medium containing, for example, water, ethanol, polyol (for example, glycerol, propylene glycol, and liquid polyethylene glycol, and the like), suitable mixtures thereof, and vegetable oils. Each carrier should be both pharmaceutically and physiologically acceptable in the sense of being compatible with the other ingredients and not injurious to the subject. Except as any conventional media or agent is incompatible with the active ingredient, its use in the pharmaceutical composition is contemplated.

In accordance with an embodiment of the present invention, the pharmaceutical composition comprising human DNase I may be administered in combination with at least one additional therapeutic agent.

The term “additional therapeutic agent” used herein refers to any agent that may be used for improving semen quality.

In certain embodiments the additional therapeutic agent is a molecule that interferes with the ability of deoxyribonucleic acid molecule to bind to receptors at the surface of sperm cells. These molecules may be molecules that bind and block the receptors or molecules that bind and block deoxyribonucleic acid molecules. Molecules that block the receptor may be of low molecular weight, for example deoxyribonucleotides dimmers, phosphodeoxyribosyl pyrophosphate (PdRPP), deoxyribonucleotides or metal ion or high molecular weight such as peptides, polypeptides, proteins, antibodies, or glycoproteins. In one embodiment, the molecule is glycoprotein IF-1 or an analog thereof capable of blocking the interaction of cell free DNA with sperm cell receptors. Molecules that bind and block deoxyribonucleic acid molecules may be peptides, polypeptides, proteins, DNA binding proteins, nuclear proteins (as histons, protamines) or antibodies. Deoxyribonucleic acid molecules may also exert a deleterious effect on sperm cells by binding at the surface of sperm cell proteins and lipids that are not receptors. In this case the agent may be a molecule that interferes with this binding.

In accordance with another embodiment of the invention, the agent may be a molecule that blocks a stage in a signal transduction pathway generated by binding of deoxyribonucleic acid molecule to receptors at the surface of sperm cells. The agent may be a low molecular weight compound or a polymer for example caspase inhibitors such as ZVAD and bcl-2 protein family or caspase dominant negative proteins.

Sperm cell damage caused by cell free DNA may involve elevated DNase activity inside the sperm cells. Thus, in accordance with another embodiment of the invention, the pharmaceutical composition of the invention may comprise a DNase inhibitor that inhibits endogenous DNase activity within the sperm cells. The agent may be a low molecular weight compound or a polymer. Preferred agents include aurintricarboxylic acid (ATA), citrate or a functional analog thereof.

In other embodiments the pharmaceutical composition according to the invention further comprises an additional therapeutic agent.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. Disclosed and described, it is to be understood that this invention is not limited to the specific examples, methods steps, and pharmaceutical compositions disclosed herein as such methods steps and pharmaceutical compositions may vary somewhat. It is also to be understood that the terminology used herein is used for the purpose of describing specific embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

### EXAMPLES

### Example 1: Pilot Study in Sub-Fertile Males

### DNase I

Recombinant human DNase I (rhDNase I, manufactured by Catalent) was supplied as a sterile solution in glass vials containing 1 mL of rhDNase I at a concentration of 10 mg/ml in formulation buffer (8.77 mg/mL of sodium chloride, 0.15 mg/mL of calcium chloride; nominal pH was 6.3).

The rhDNase I enzyme is identical to the endogenous human enzyme (CAS #: 0143831-71-4).

The amino acid sequence of human DNase I (UniProt, Human DNase-I, Isoform 1, identifier: P24855-1) is outlined in SEQ ID NO:1.

The rhDNase was produced by genetically engineered Chinese hamster ovary (CHO) cells containing DNA that encodes for the human protein, deoxyribonuclease I (DNase). Purification of the product is achieved by conventional tangential flow filtration and column chromatography technology. The purified glycoprotein contains 260 amino acids with a relative molecular weight of approximately 37,000 daltons.

### The Clinical Study

The study was a prospective, open-label, non-controlled, single-center study to evaluate the efficacy of Intravenous (IV) Injections of rhDNase I in sub-fertile male patients.

Diagnosis and Main Criteria for Inclusion: Male partners of sub-fertile couples that had been attempting to conceive for more than two years and had previous failures in assisted reproductive technology (ART) with no diagnosed female factor of infertility. The male patient was 18 to 50 years of age and had documented idiopathic reduction in semen quality but not azoospermia. One subject with azoospermia was enrolled in a deviation from the study protocol.

Study Endpoints: Evidence of improvement in one or more semen quality parameters of sub-fertile men following rhDNase I treatment. Semen quality parameters assessed: total count, concentration, motility, motility quality or average speed, morphology (Kruger or WHO), viability (eosin staining), fine morphology according to MSOME, sperm chromatin stability (Halosperm assay).

All subjects received two daily Intravenous injections of 125 μg/kg body weight rhDNase I on days 1-16. The injections were given in the morning and in the evening—12 hours apart.

The study drug was provided as 1 mg/ml rhDNase I, 2.5 ml per vial.

Four subjects participated in the study. Subject #001 had Azoospermia, subject #002 had Severe Isolated Teratozoospermia (100% amorphic heads), subject #003 had Mild Isolated Teratozoospermia (12% Normal forms, according to World Health Organization (WHO) definitions) and subject #004 had Severe Isolated Teratozoospermia.

Safety results: No abnormalities in vital signs and physical examination findings were observed. No adverse events were reported. No deaths or serious adverse events occurred during the study.

Efficacy results: Improvements in semen quality were seen in 3 out of the 4 treated subjects. Three out of the four couples had successful pregnancies using semen samples taken following rhDNase I treatment.

### Subject #001:

Baseline characteristics:
Azoospermic patient.
High follicle-stimulating hormone (FSH) levels.
Attempting to conceive or miscarrying for 4.5 years.
Failed in 2 intracytoplasmic sperm injection (ICSI) cycles.
Failed in 3 intracytoplasmic morphologically selected sperm injection (IMSI) cycles, one of them resulted in an abortion in the 13th week.
Suffered from left testicular varicocele which was operated in 2001.
High blood cell free DNA levels.

The patient was treated with rhDNase I as specified above and the semen quality was analyzed. Due to the subject's Azoosperima, the only semen parameter which could be followed was High power (×6,000) motile sperm organelle morphology examination (MSOME, Bartoov B, et al. J Androl. 2002 January-February; 23(1): 1-8)) of the small number of sperm cells observed.

### Sperm Preparation for Morphological Observation in Wet (Regular) MSOME:

An aliquot of 1-2 μl of the sperm suspension containing a few thousand spermatozoa was transferred to a microdroplet of sperm medium containing 0%-8% polyvinyl pyrrolidone (PVP) solution and placed in a glass-bottom dish under paraffin oil. Morphological assessment of the sperm cells in motion was made possible by the creation of small bays extending from the rim of the droplets, which captured the heads of the motile spermatozoa.

### Sperm Preparation for Morphological Observation in Dry MSOME:

Fresh ejaculate post liquefaction was transferred to 15 ml round bottom tubes at room temperature (22-25° C.). The soft pellet of ejaculated sperm cells was obtained by centrifugation in swing-out buckets at 1500 RPM for 15 minutes at room temperature. The seminal plasma supernatant was removed. The sperm pellet was suspended in 1 ml Ferticult-IVF medium (FertiPro N.V. Beernem, Belgium) and then re-centrifuged at 1500 RPM for 5 minutes at room temperature. The tubes were then transferred to a 5 percent CO₂, 37° C. incubator for a 40-minute swim-up incubation, inclined at 45°. Supernatant was then carefully removed until the interphase of the sperm pellet. The swim-up supernatant was centrifuged at 1500 RPM for 5 minutes at room temperature and the supernatant was discarded. The pellet was smeared on microscope glass slides and air dried.

### Sperm MSOME Observation:

Sperm cells were examined at 21° C. by an inverted microscope (Olympus IX 81) equipped with Nomarski optics, an Uplan Apo X 100/1.35 objective lens, and a 0.55 NA condenser lens. The images were captured by a DXC-950P color video camera (Sony). The morphological assessment was conducted on the monitor screen which, under the above configuration, reached a real magnification of 6300.

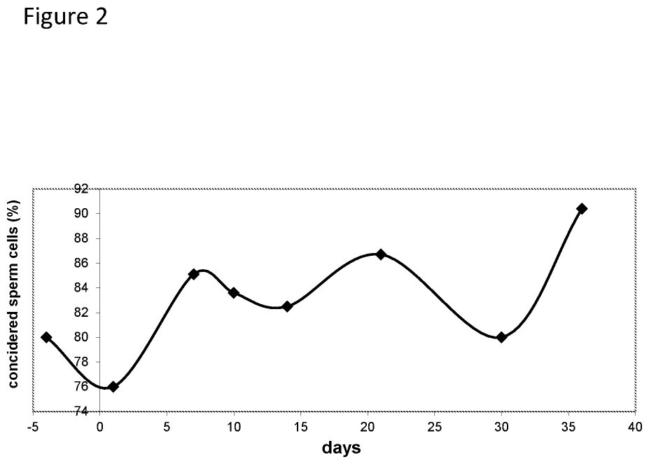

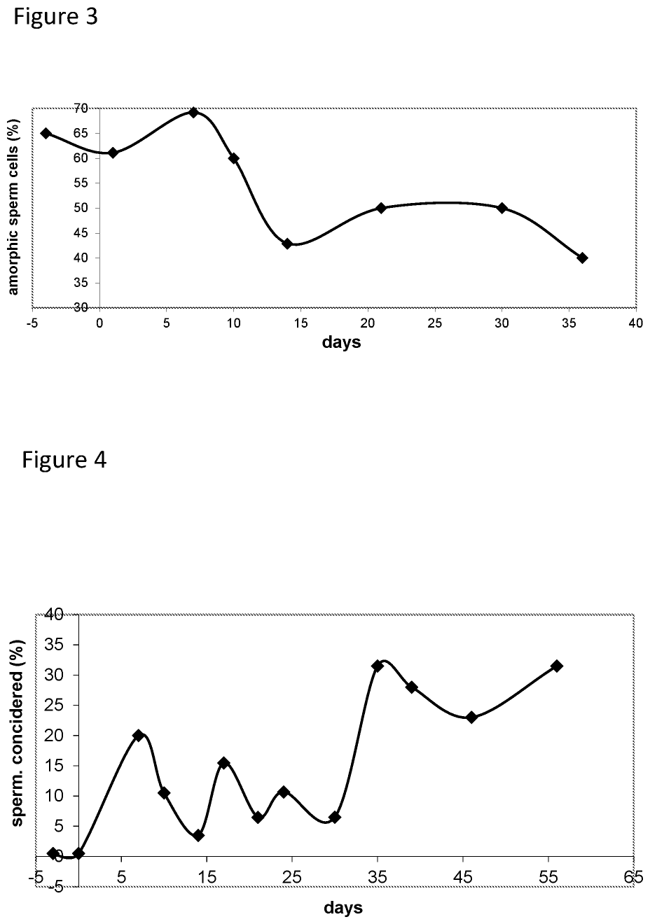

The results of the MSOME are provided in FIG. 2 and FIG. 3. The results show elevation in sperm cells with oval shape head (FIG. 2) which represent normal sperm cell morphology and reduction in amorphous shape sperm heads (FIG. 3) which represent abnormal sperm cell morphology. The rectangle represents the 16-day period of DNase I treatment. Only sperm cells without rough morphological malformations were included in this analysis.

### Subject #002:

Baseline characteristics:
Normal semen volume (˜2 ml).
Normal sperm concentration (˜35 Million/ml).
Normal sperm density (˜ 70 Million/ejaculate).
Normal sperm motility (˜50%).
Normal viability (˜70%).
Very Severe Isolated Teratozoospermia (100%).
Attempting to conceive for 8 years.
Failed in 4 ICSI cycles.
No fertilizations in IVF (in vitro fertilization)
Suffered from testicular left varicocele.
High blood cell-free DNA levels.

Measurement of semen volume and sperm cells concentration, density, motility, and viability was performed according to WHO guidelines (see WHO laboratory manual for the examination of human semen and sperm-cervical mucus interaction. Fourth edition 1999. World Health Organization. Cambridge university press).

Briefly, the volume of each semen ejaculate was measured using a pipette. Sperm concentration was determined by counting the cells using either Helber hemocytometer or Neubauer hemocytometer (Helber is used when sperm concentration is high and Neubaure when it is low). Sperm density (=cell number in whole ejaculate) was calculated by multiplying the ejaculate volume by sperm concentration.

Sperm Motility and Progressive Motility: Motile and non-motile sperm cells were counted using either Helber hemocytometer or Neubauer hemocytometer and the percent of motile sperm cells was calculated accordingly. A sample of 20 random motile sperm cells was examined for progressive motility by examining their ability to progress in a straight line through 200 μm and the percentage of progressively motile sperm cell was calculated accordingly.

rhDNase I treatment had no effect on sperm concentration, density and sperm cell viability and motility (not shown) which were all in the normal range. However, as demonstrated in the FIGS. 4-6, rhDNase I treatment had beneficial effects on the following parameters:

1. Sperm morphology, as manifested by elevation (from 0% to 30%) in sperm cells with oval shape heads and by reduction in the number of sperm cells with round and amorphous shape heads.

2. Sperm DNA stability was measured by the Halosperm test that allows the measurement of DNA fragmentation. Halosperm is based on the Sperm Chromatin Dispersion (SCD) technique. The Halosperm test is an in vitro diagnostic kit [Halotech DNA, Spain] that controlled DNA denaturation process to facilitate the subsequent removal of the proteins contained in each spermatozoon. In this way, normal spermatozoa create halos formed by loops of DNA at the head of the sperm, which are not present in those with damaged DNA. As indicated by the Halosperm test, the percent of sperm cells without DNA fragmentation increased from 10% to 50%.

3. Three pregnancies were achieved using semen obtained following the rhDNase I treatment.

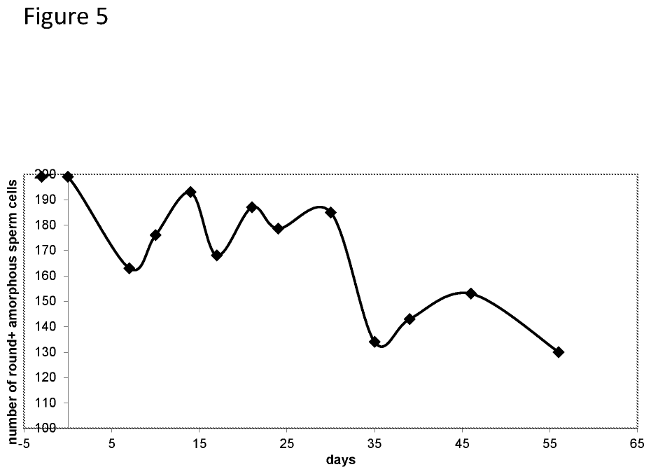

DNase I treatment resulted in an elevation in sperm cells with oval shape heads (FIG. 4), and a reduction in the number of sperm cells with round and amorphous shape heads (FIG. 5). The rectangle represents the 16-day period of DNase I treatment. Only sperm cells without rough morphological malformations were included in this analysis.

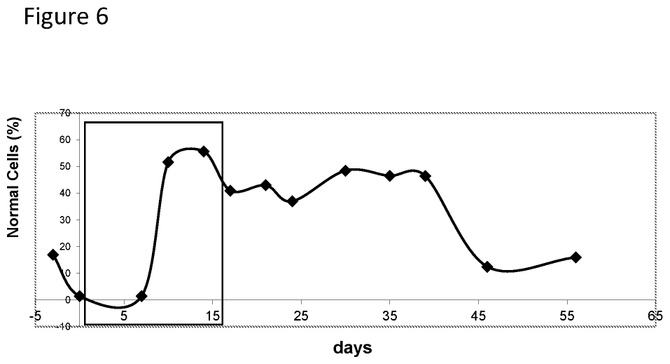

As can be seen in FIG. 6, treatment with rhDNase I (at days 1-16 as marked by the rectangle) caused an elevation in the percent of normal sperm cells without DNA fragmentation, as demonstrated by Halosperm analysis.

### Subject #003:

Baseline characteristics:
Low semen volume (˜1 ml)
Normal sperm concentration (˜50 Million/ml)
Normal sperm cell density (˜ 50 Million/ejaculate)
Normal sperm cells motility (˜60%)
Normal sperm cells viability (˜75%)
Mild Isolated Teratozoospermia (12% in WHO, 2% by Kruger's criteria)
Attempting to conceive for 2 years.

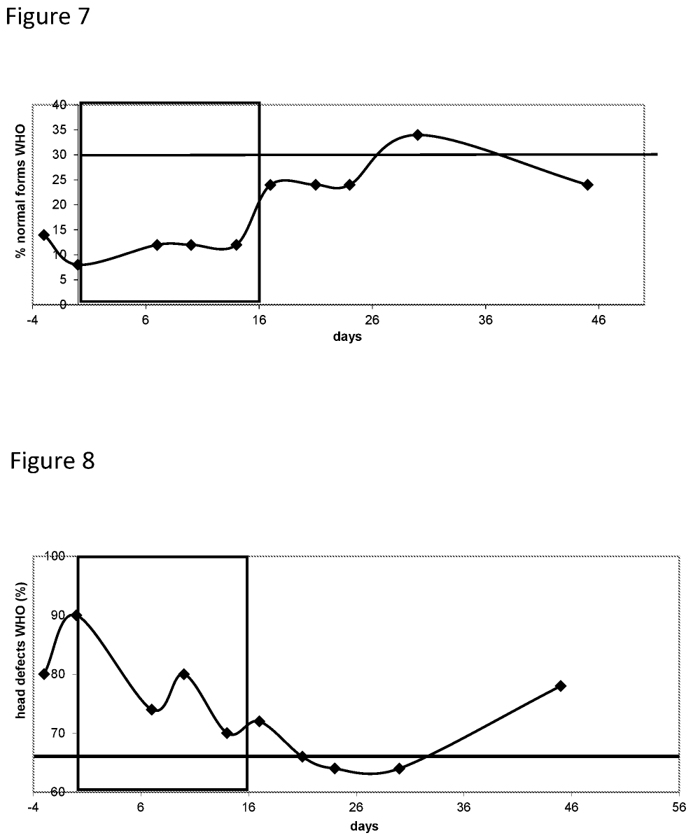

rhDNase I treatment did not induce major changes in sperm concentration, density and sperm cells viability and motility (not shown), which were all in the normal range. Rhythmic changes in semen volume were observed, possibly due to semen hypovolemia. However, as demonstrated in the FIGS. 7-9, rhDNase I treatment had beneficial effects on the following parameters:
1. Sperm morphology, as manifested by a 3-fold increase in normal forms according to the WHO 4th edition criteria (reaching normal values), 30% decrease in sperm head malformations according to WHO criteria (reaching normal values) and 5-fold increase in the percent of sperm cells with normal nucleus as observed in MSOME (reaching borderline values).
2. Sperm DNA stability, as measured by the Halosperm test (a 2.5-fold increase in sperm cells without DNA fragmentation, reaching normal values).
3. One pregnancy was achieved using semen obtained following rhDNase I treatment.

These changes demonstrate that subject #003 has achieved Normozoospermia following the treatment with rhDNase I.

As can be seen in FIG. 7, treatment with rhDNase I (at days 1-16 as marked by the rectangle) caused an improvement in sperm morphology according to WHO criteria.

As can be seen in FIG. 8, treatment with rhDNase I (at days 1-16 as marked by the rectangle) led to a decrease in sperm head malformations according to WHO criteria.

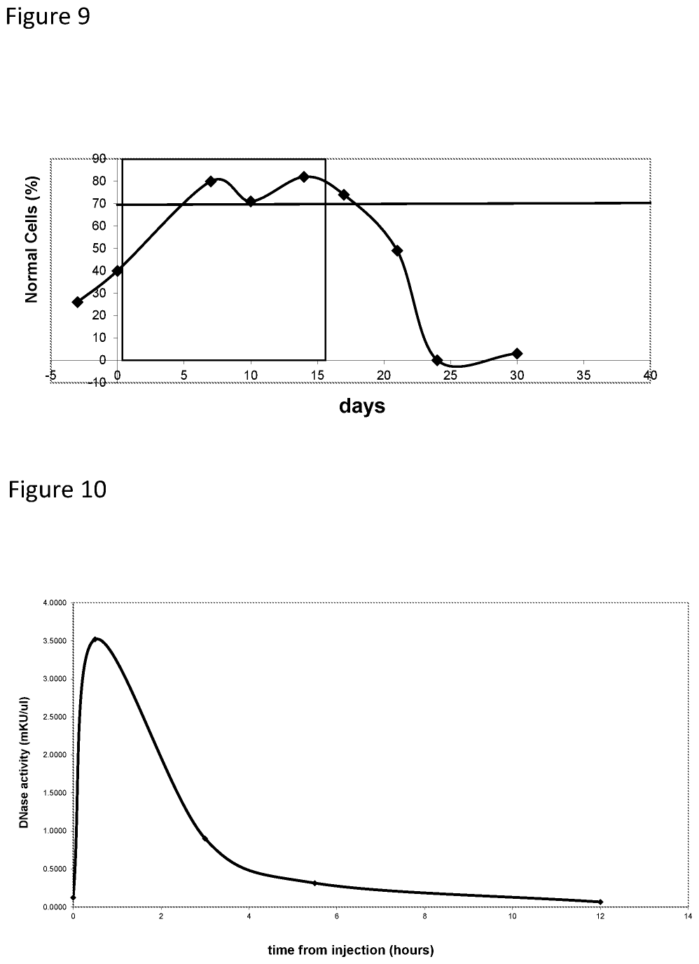

As can be seen in FIG. 9, treatment with rhDNase I (at days 1-16 as marked by the rectangle) led to an elevation in percent of sperm cells without DNA fragmentation.

### Subject #004

Baseline characteristics:
Normal semen volume (˜2 ml)
low sperm concentration (˜13 Million/ml)
Severe sperm Motility (˜12%)
Severe viability (˜33%)
Very Severe Isolated Teratozoospermia (100%)
High blood cell-free DNA levels.
Attempting to conceive for 2.5 years.
Failure in one intrauterine insemination (IUI)
Failure in 2 ICSI cycles

No significant changes in sperm cells were observed following DNase I treatment; nevertheless, one pregnancy was achieved using semen obtained in the study.

The DNase activity in the serum of treated subjects after injection of 125 μg/kg rhDNase was assessed using the Single Radial Enzyme Diffusion (SRED) test performed according to Nadano D, et al. Clin Chem. 1993 March; 39(3):448-52. Analysis of one representative subject (Subject #001) is provided in FIG. 10.

1.-32. (canceled)

33. A method for induction of pregnancy in a couple in need thereof wherein the male subject in said couple has been diagnosed as sub-fertile, said method comprising administering systemically to said male subject a pharmaceutical composition comprising between about 50 and about 1000 μg/kg human DNase, for a treatment course period.

34. The method of claim 33 wherein said pharmaceutical composition is administered for a treatment course period sufficient to achieve fresh spermatid maturation in said male subject.

35. The method of claim 33 wherein said administration is for a treatment course period of between about 10 days and about 3 months, or between about 10 days and 16 days.

36. The method of claim 33, wherein the human male subject has an idiopathic reduction in semen quality, a low sperm viability and/or motility and/or concentration, azoospermia, oligozoospermia, mild or severe isolated teratozoospermia, asthenozoospermia, or oligoteratoasthenospermia (OTA).

37. The method of claim 33, wherein the human male subject is a male partner of a couple that had been failing to conceive or had been miscarrying for two years or more or had been failing to conceive in at least one assisted reproductive technology (ART) procedure.

38. The method of claim 37 wherein the human female subject of said couple was not diagnosed with infertility.

39. The method of claim 33 wherein the sperm of said human male subject is used in ART.

40. The method of claim 33 wherein administration of said pharmaceutical composition results in improvement of at least 35% in one or more sperm quality parameters selected from the group consisting of total sperm count, sperm concentration, sperm motility, sperm motility quality or average speed, sperm morphology, sperm viability and sperm chromatin stability.

41. The method of claim 33 wherein the sperm of said human male subject is sampled within 1 to 14 days following completion of said treatment course period.

42. The method of claim 33 wherein the sperm of said human male subject is sampled within 1 to 5 days following completion of said treatment course period.

43. The method of claim 33 wherein completion of the treatment course period with said pharmaceutical composition is synchronized with ovulation in the female subject in said couple.

44. The method of claim 43 wherein ovulation subsides 1 to 14 days following completion of the treatment course period.

45. The method of claim 43 wherein ovulation subsides 1 to 5 days following completion of the treatment course period.

46. The method of claim 33 further comprising administration of an additional therapeutic agent.

47. The method of claim 33 wherein said DNase is pegylated DNAse or PASylated DNAse.

48. The method of claim 33, wherein said DNase is human DNase 1.

49. The method of claim 48 wherein said human DNAse I comprises an amino acid sequence having at least 90% sequence identity to SEQ ID NO: 1 or to the mature DNAse I enzyme without the signal peptide.

50. The method of claim 48 wherein said DNase I is human recombinant DNase I, or isolated natural human DNase I.

51. The method of claim 33 wherein said DNase I is human DNase I, comprising an amino acid sequence having at least 90% sequence identity to SEQ ID NO: 1, and wherein said human DNase I is injected intravenously for 14-16 days at a dose of 250 μg/kg/day and wherein the sperm of said human male subject is sampled within 1 to 5 days following completion of said treatment course period.

52. The method of claim 33 wherein said DNase I is human DNase I, comprising an amino acid sequence having at least 90% sequence identity to SEQ ID NO: 1, and wherein said human DNase I is injected intravenously for 14-16 days at the dose of 250 μg/kg/day and wherein ovulation subsides 1 to 5 days following completion of the treatment course period.
